(19) United States
(12) Patent Application Publication
(10) Pub. No.: US 2009/0175824 A1
(43) Pub. Date: Jul. 9, 2009

(54) Peptides for the treatment of HCV infections

(76) Inventor: Craig Masse, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/313,507

(22) Filed: Nov. 20, 2008

Related U.S. Application Data
(60) Provisional application No. 61/003,857, filed on Nov. 20, 2007.

(51) Int. Cl. A61K 38/21; C07K 5/083; A61K 38/06; A61P 31/12

(52) U.S. Cl. 424/85.7; 530/331; 514/18

(57) ABSTRACT

This invention relates to novel compounds that are peptides derivatives and pharmaceutically acceptable salts thereof. More specifically, this invention relates to novel peptides that are derivatives of boceprevir. This invention also provides compositions comprising one or more compounds of this invention and a carrier and the use of the disclosed compounds and compositions in methods of treating diseases and conditions that are beneficially treated by administering an HCV NS3/NS4A protease inhibitor, such as boceprevir.

### RELATED APPLICATION

This application claims the benefit of U.S. Provisional Application No. 61/003,857, filed on Nov. 20, 2007. The entire teachings of the above application is incorporated herein by reference.

Boceprevir, also known as SCH-503034, or as N-(4-amino-1-cyclobutyl-3,4-dioxobutan-2-yl)-3-(2-(3-tert-butylureido)-3,3-dimethylbutanoyl)-6,6-dimethyl-3-azabicyclo[3.1.0]hexane-2-carboxamide, inhibits HCV NS3/NS4a serine protease. HCV is a (+)-sense single-stranded RNA virus that has been implicated as the major causative agent in non-A, non-B hepatitis (NANBH). Of the several non-structural proteins (NS1, NS2, NS3, NS4a, NS5a, and NS5b) contained in the HCV protease, HCV NS3 serine protease is responsible for proteolysis of the polypeptide at the NS3/NS4a, NS4a/NS4b, NS4b/NS5a, and NS5a/NS5b junctions and is thus responsible for generating five viral proteins during viral replication. The NS4a protein is a co-factor for the serine protease activity of NS3. (See International Patent Publication no. WO 2002008244).

Boceprevir is currently in phase III clinical trials for treatment of hepatitis C.

In general, boceprevir is well-tolerated in patients with hepatitis C. Adverse events were infrequent and included headache, fever and myalgia. (Sarrazin, C et al, Gastroenterology, 2007, 132(4): 1270 and Zeuzem, S et al, 56th Annu Meet Am Assoc Study Liver Dis, 2005, (November 11-15, San Francisco): Abst 201).

Despite the beneficial activities of boceprevir, there is a continuing need for new compounds to treat hepatitis C.

### SUMMARY OF THE INVENTION

This invention relates to novel compounds that are peptide derivatives and pharmaceutically acceptable salts thereof. More specifically, this invention relates to novel peptides that are derivatives of boceprevir. This invention also provides pharmaceutical compositions comprising one or more compounds of this invention and a pharmaceutically acceptable carrier and the use of the disclosed compounds and compositions in methods of treating diseases and conditions that are beneficially treated by administering an HCV NS3/NS4A protease inhibitor, such as boceprevir.

### DETAILED DESCRIPTION OF THE INVENTION

The terms “ameliorate” and “treat” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of boceprevir will inherently contain small amounts of deuterated isotopologues. The concentration of naturally abundant stable hydrogen isotopes, notwithstanding this variation, is small and immaterial as compared to the degree of stable isotopic substitution of compounds of this invention. See, for instance, Wada, E et al, Seikagaku, 1994, 66: 15; Ganes, L Z et al, Comp Biochem Physiol Mol Integr Physiol, 1998, 119: 725.

The compounds of the present invention are distinguished from such naturally occurring minor forms in that the term “compound” as used in this invention refers to a composition of matter that has a minimum isotopic enrichment factor at least 3000 (45% deuterium incorporation) for each deuterium atom that is present at a site designated as a site of deuteration in Formula (I).

In the compounds of the invention, any atom not specifically designated as a particular isotope is meant to represent any stable isotope of that atom unless otherwise stated. Unless otherwise stated, when a position is designated specifically as “H” or “hydrogen,” the position is understood to have hydrogen at its natural abundance isotopic composition. Also unless otherwise stated, when a position is designated specifically as “D” or “deuterium”, the position is understood to have deuterium at an abundance at least 3000 times the natural abundance of deuterium, which is 0.015% (i.e., at least 45% deuterium incorporation).

The term “isotopic enrichment factor” as used herein means the ratio between the isotopic abundance and the natural abundance of a specified isotope.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The structural formula depicted herein may or may not indicate whether atoms at certain positions are isotopically enriched. In a most general embodiment, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, the stable isotopes are present at natural abundance at all positions in a compound not specifically designated as being isotopically enriched.

The term “isotopologue” refers to a species that differs from a specific compound of this invention only in the isotopic composition thereof. Isotopologues can differ in the level of isotopic enrichment at one or more positions and/or in the positions(s) of isotopic enrichment.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 55% of the compound. In other embodiments, the relative amount of such isotopologues in toto will be less than 50%, less than 47.5%, less than 40%, less than 32.5%, less than 25%, less than 17.5%, less than 10%, less than 5%, less than 3%, less than 1%, or less than 0.5% of the compound.

The invention also provides salts, solvates or hydrates of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refer to deuterium. “Stereoisomer” refers to both enantiomers and diastereomers.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

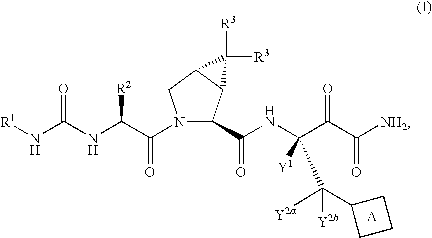

or a pharmaceutically acceptable salt thereof, wherein:

Ring A is a cyclobutyl ring having 0-7 deuterium atoms;

each of R¹ and R² is independently —C(CH₃)₃, wherein 1 to 9 hydrogen atoms are optionally replaced with deuterium atoms;

each R³ is independently selected from —CH₃, —CH₂D, —CHD₂, and —CD₃;

each Y is independently selected from hydrogen and deuterium; and

at least one Y is deuterium when R¹ and R² are simultaneously —C(CH₃)₃, R³ is —CH₃, and ring A has zero deuterium atoms.

In one embodiment of a compound of Formula I:

Ring A is a cyclobutyl ring having either 0 deuterium atoms or 7 deuterium atoms;

R¹ and R² are the same;

each R³ is the same; and

each Y is the same.

In another embodiment of a compound of Formula I:

Ring A is a cyclobutyl ring having either 0 deuterium atoms or 7 deuterium atoms;

R¹ and R² is the same and are selected from C(CH₃)₃ and —C(CD₃)₃; and

each R³ is the same and is selected from —CH₃, and —CD₃.

Other embodiments of a compound of formula I include those wherein:

a) R¹ is selected from —C(CH₃)₃ and —C(CD₃)₃;

b) R² is selected from —C(CH₃)₃ and —C(CD₃)₃;

c) each R³ is the same;

d) each R³ is independently selected from —CH₃ and —CD₃;

e) Ring A is a cyclobutyl ring having 0 or 7 deuterium atoms; or

f) each Y² is the same.

In another embodiment, a compound of Formula I has the characteristics set forth in one or more of a) through f), above; and at least one of R¹ and R² is —C(CD₃)₃, or each R³ is —CD₃. For example, at least one of R¹ and R² is —C(CD₃)₃, or each R³ is —CD₃ and Ring A is a cyclobutyl ring having 0 or 7 deuterium atoms or at least one of R¹ and R² is —C(CD₃)₃; or each R³ is —CD₃ and each Y² is the same.

In still another embodiment, a compound of Formula I has the characteristics set forth in two or more of a) through f), above. Such combinations include, but are not limited to: a and b; a and c; b and c; a, b and c; a and d; b and d; a, b and d; a, c and d; b, c, and d; a, b, c and d; e and c; e and d; e and a; e and b; e, b and a; e, c and a; e, c and b; e, c, b and a; e, d and a; e, d and b; e, d, b and a; e, d, c and a; e, d, c and b; e, d, c, b and a; a and f; b and f; a, b and f; c and f; a, c, and f; b, c and f; a, b, c and f; d and f; b and f; a, b, d and f; a, c, d and f; b, c, d and f; a, b, c, d, and f.

In still another embodiment, each of R¹ and R² is —C(CD₃)₃, each R³ is the same and is selected from —CD₃ and —CH₃, each Y² is the same, and Ring A is selected from a cyclobutyl ring having 0 deuterium atoms and a cyclobutyl ring having 7 deuterium atoms.

Examples of specific compounds of Formula I are shown in Table 1 below. In these examples, Y²a is the same as Y²b and Ring A has zero deuterium atoms (H₇) or seven deuterium atoms (D₇) replacing hydrogen atoms at available ring carbon positions.

TABLE 1
Examples of Specific Compounds of Formula I

| Compound | R¹ | R² | each R³ | Y¹ | each Y² | Ring A |
|---|---|---|---|---|---|---|
| 100 | C(CD₃)₃ | C(CD₃)₃ | CD₃ | D | D | D₇ |
| 101 | C(CD₃)₃ | C(CD₃)₃ | CD₃ | D | D | H₇ |
| 102 | C(CD₃)₃ | C(CD₃)₃ | CD₃ | D | H | H₇ |
| 103 | C(CD₃)₃ | C(CD₃)₃ | CD₃ | H | H | H₇ |
| 104 | C(CD₃)₃ | C(CD₃)₃ | CD₃ | H | H | D₇ |
| 105 | C(CD₃)₃ | C(CD₃)₃ | CD₃ | D | H | D₇ |
| 106 | C(CD₃)₃ | C(CD₃)₃ | CH₃ | D | D | D₇ |
| 107 | C(CD₃)₃ | C(CD₃)₃ | CH₃ | H | D | D₇ |
| 108 | C(CD₃)₃ | C(CD₃)₃ | CH₃ | H | H | D₇ |
| 109 | C(CD₃)₃ | C(CD₃)₃ | CH₃ | H | H | H₇ |
| 110 | C(CD₃)₃ | C(CD₃)₃ | CH₃ | H | D | H₇ |
| 111 | C(CD₃)₃ | C(CD₃)₃ | CH₃ | D | D | H₇ |
| 112 | C(CD₃)₃ | C(CD₃)₃ | CH₃ | D | H | H₇ |
| 113 | C(CD₃)₃ | C(CH₃)₃ | CD₃ | D | D | D₇ |
| 114 | C(CD₃)₃ | C(CH₃)₃ | CD₃ | H | D | D₇ |
| 115 | C(CD₃)₃ | C(CH₃)₃ | CD₃ | H | H | D₇ |
| 116 | C(CD₃)₃ | C(CH₃)₃ | CD₃ | D | D | H₇ |
| 117 | C(CD₃)₃ | C(CH₃)₃ | CD₃ | H | D | H₇ |
| 118 | C(CD₃)₃ | C(CH₃)₃ | CD₃ | D | H | H₇ |
| 119 | C(CD₃)₃ | C(CH₃)₃ | CD₃ | H | H | H₇ |
| 120 | C(CH₃)₃ | C(CD₃)₃ | CD₃ | D | D | D₇ |
| 121 | C(CH₃)₃ | C(CD₃)₃ | CD₃ | H | D | D₇ |
| 122 | C(CH₃)₃ | C(CD₃)₃ | CD₃ | H | H | D₇ |
| 123 | C(CH₃)₃ | C(CD₃)₃ | CD₃ | D | H | D₇ |
| 124 | C(CH₃)₃ | C(CD₃)₃ | CD₃ | D | D | H₇ |
| 125 | C(CH₃)₃ | C(CD₃)₃ | CD₃ | H | D | H₇ |
| 126 | C(CH₃)₃ | C(CD₃)₃ | CD₃ | H | H | H₇ |
| 127 | C(CH₃)₃ | C(CD₃)₃ | CD₃ | D | H | H₇ |
| 128 | C(CH₃)₃ | C(CH₃)₃ | CD₃ | D | D | D₇ |
| 129 | C(CH₃)₃ | C(CH₃)₃ | CD₃ | D | H | D₇ |
| 130 | C(CH₃)₃ | C(CH₃)₃ | CD₃ | H | D | D₇ |
| 131 | C(CH₃)₃ | C(CH₃)₃ | CD₃ | D | D | H₇ |
| 132 | C(CH₃)₃ | C(CH₃)₃ | CD₃ | H | D | H₇ |
| 133 | C(CH₃)₃ | C(CH₃)₃ | CD₃ | H | H | D₇ |
| 134 | C(CH₃)₃ | C(CH₃)₃ | CD₃ | H | H | H₇ |
| 135 | C(CH₃)₃ | C(CH₃)₃ | CD₃ | D | H | H₇ |
| 136 | C(CD₃)₃ | C(CH₃)₃ | CH₃ | D | D | D₇ |
| 137 | C(CD₃)₃ | C(CH₃)₃ | CH₃ | D | H | D₇ |
| 138 | C(CD₃)₃ | C(CH₃)₃ | CH₃ | H | D | D₇ |
| 139 | C(CD₃)₃ | C(CH₃)₃ | CH₃ | D | D | H₇ |
| 140 | C(CD₃)₃ | C(CH₃)₃ | CH₃ | H | D | H₇ |
| 141 | C(CD₃)₃ | C(CH₃)₃ | CH₃ | H | H | D₇ |
| 142 | C(CD₃)₃ | C(CH₃)₃ | CH₃ | D | H | H₇ |
| 143 | C(CD₃)₃ | C(CH₃)₃ | CH₃ | H | H | H₇ |
| 144 | C(CH₃)₃ | C(CD₃)₃ | CH₃ | D | D | D₇ |
| 145 | C(CH₃)₃ | C(CD₃)₃ | CH₃ | D | H | D₇ |
| 146 | C(CH₃)₃ | C(CD₃)₃ | CH₃ | H | D | D₇ |
| 147 | C(CH₃)₃ | C(CD₃)₃ | CH₃ | D | D | H₇ |
| 148 | C(CH₃)₃ | C(CD₃)₃ | CH₃ | H | D | H₇ |
| 149 | C(CH₃)₃ | C(CD₃)₃ | CH₃ | H | H | D₇ |
| 150 | C(CH₃)₃ | C(CD₃)₃ | CH₃ | D | H | H₇ |
| 151 | C(CH₃)₃ | C(CD₃)₃ | CH₃ | H | H | H₇ |
| 152 | C(CH₃)₃ | C(CH₃)₃ | CH₃ | D | D | D₇ |
| 153 | C(CH₃)₃ | C(CH₃)₃ | CH₃ | D | H | D₇ |
| 154 | C(CH₃)₃ | C(CH₃)₃ | CH₃ | H | D | D₇ |
| 155 | C(CH₃)₃ | C(CH₃)₃ | CH₃ | D | D | H₇ |
| 156 | C(CH₃)₃ | C(CH₃)₃ | CH₃ | H | D | H₇ |
| 157 | C(CH₃)₃ | C(CH₃)₃ | CH₃ | H | H | D₇ |

or a pharmaceutically acceptable salt of any of the foregoing.

In yet another embodiment, the compound is selected from:

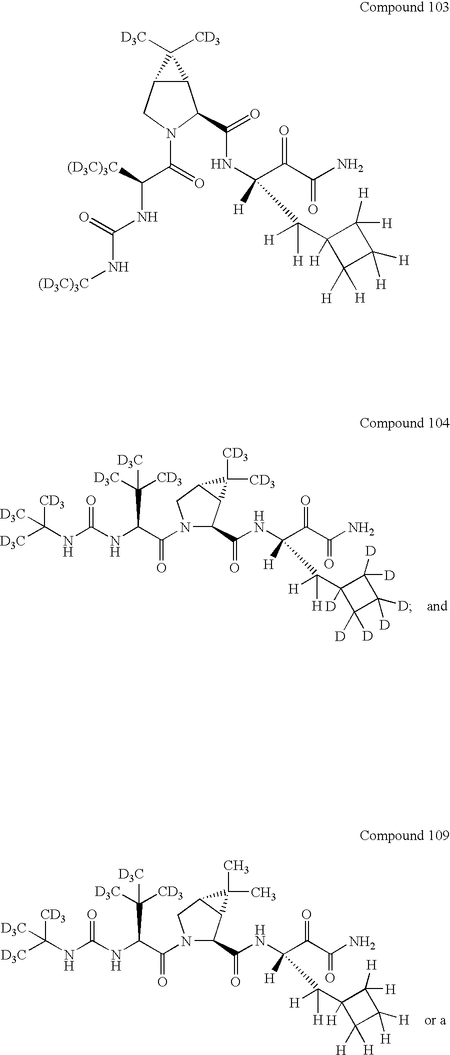

pharmaceutically acceptable salt of any of the foregoing.

In another set of embodiments, any atom not designated as deuterium in any of the embodiments set forth above is present at its natural isotopic abundance.

The synthesis of compounds of Formula I can be readily achieved by synthetic chemists of ordinary skill. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in PCT publication WO 2004/113294, United States Patent publication US20070004635, or in Venkatraman, S et al, J Med Chem 2006, 49:6074. The compounds may be prepared as illustrated in the schemes shown below.

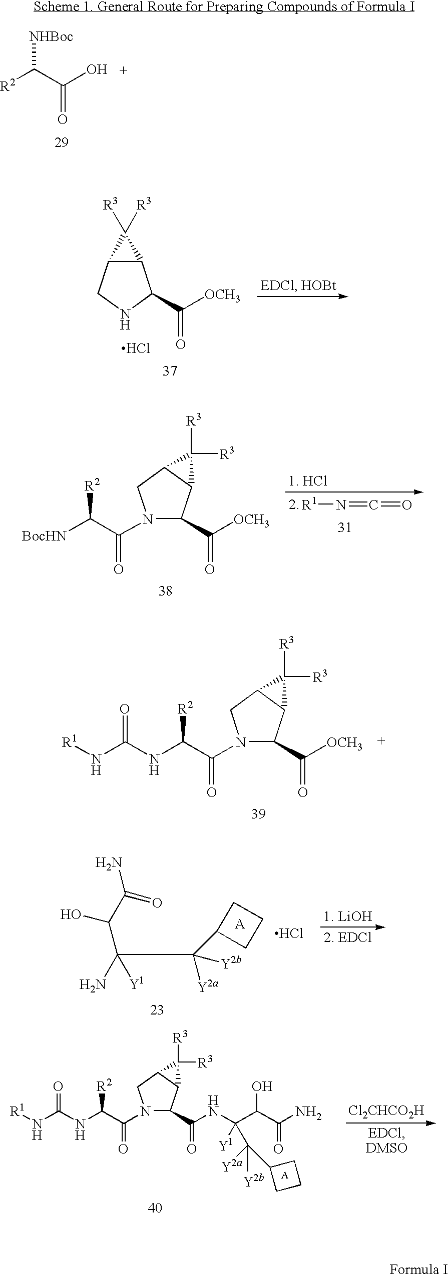

Scheme 1 above shows a general route for preparing compounds of Formula I. Deuterated 3,4-dimethylcyclopropylproline 37 is condensed with deuterated N-Boc-tert-leucine reagent 29 using the procedures described by Venkatraman, S et al., J Med Chem 2006, 49: 6074-6086 to afford the dipeptide 38. Acidic removal of the Boc group with HCl and reaction of the corresponding amine with deuterated tert-butylisocyanate 31 (prepared from the correspondingly deuterated amine R¹—NH₂ 30 by treatment with hydrochloric acid and then triphosgene) affords the dipeptide 39. Final coupling to alpha-hydroxy amide 23 produces hydroxyamide 40, which is then oxidized to afford a compound of Formula I.

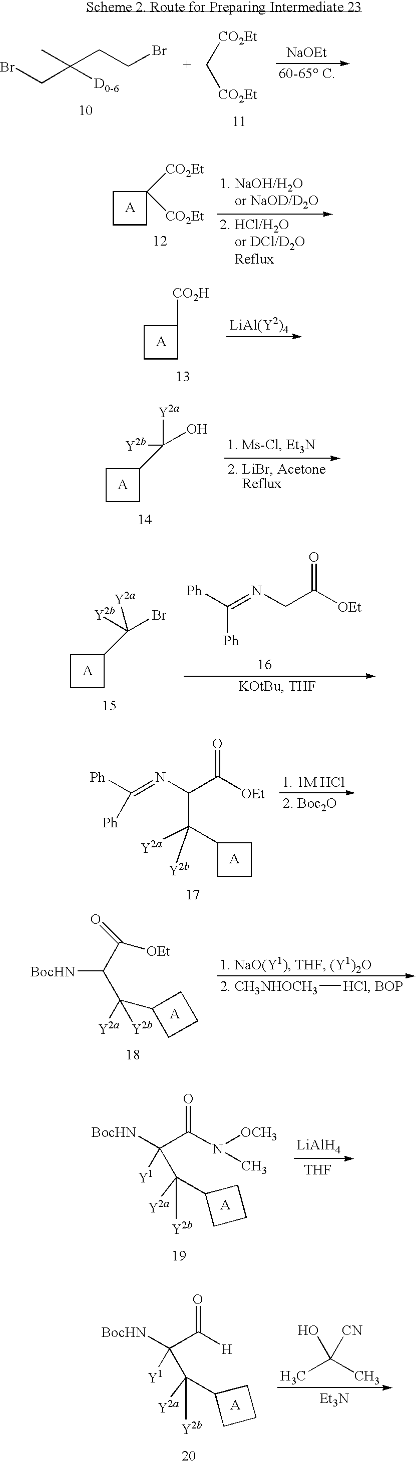

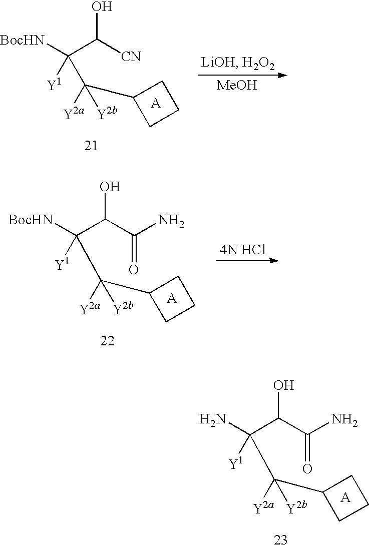

Scheme 2 shows a route for preparing deuterated cyclobutylmethyl alpha-hydroxy amide 23, which is useful in Scheme 1. Treatment of a deuterated dibromopropane 10 (such as commercially available dibromopropane-d₆) with diethylmalonate 11 in the presence of sodium ethoxide using the procedure described by Heisig, G B et al, Org Synth 1955, 3:213-215 affords the corresponding ethyl-1,1-cyclobutanedicarboxylate 12. Saponification of the ethyl ester moieties in 12 with sodium hydroxide or sodium deuteroxide followed by decarboxlyation with hydrochloric acid or deuterium chloride using the procedures in the aforementioned reference affords deuterated cyclobutanecarboxylic acid 13. Reduction of 13 with lithium aluminum deuteride or lithium aluminum hydride (LiAl(Y²)₄) using the procedure described by Ingold, K U, et al, J Chem Soc, Perkin Trans II 1981, pp 970-974 affords deuterated cyclobutylmethanol 14. Subsequent activation of the alcohol moiety in 14 as the corresponding mesylate and displacement with lithium bromide using the procedure from Ingold affords the corresponding cyclobutylmethyl bromide 15.

The cyclobutylmethyl bromide 15 is then combined with the potassium enolate of ethyl N-(diphenylmethylene)glycinate 16 (generated in situ by treatment of 16 with potassium tert-butoxide) using the protocol described by Venkatraman, S et al, J Med Chem 2006, 49:6074-6086 to afford the corresponding glycine derivative 17. Hydrolysis of the diphenylimine moiety in 17 followed by Boc protection of the amine produces the Boc-protected amino ester 18. The Y¹ group is incorporated by treatment of 18 with either sodium hydroxide or sodium deuteroxide (NaOY¹), followed by conversion of the resulting acid to the corresponding Weinreb amide 19 upon treatment with N,O-dimethylhydroxylamine and benzotriazole-1-yl-oxy-tris-(dimethylamino)-phosphonium hexafluorophosphate (BOP). Conversion of amide 19 to the corresponding aldehyde 20 is achieved by treatment with LiAlH₄ in THF. Aldehyde 20 is treated with acetone cyanohydrin in triethylamine to produce cyanohydrin 21. Hydrolysis of cyanohydrin 21 to hydroxyamide 22 is accomplished by treatment with LiOH and hydrogen peroxide. This is followed by acid catalyzed cleavage of the Boc group in 22 to form a deuterated alpha hydroxy amide 23.

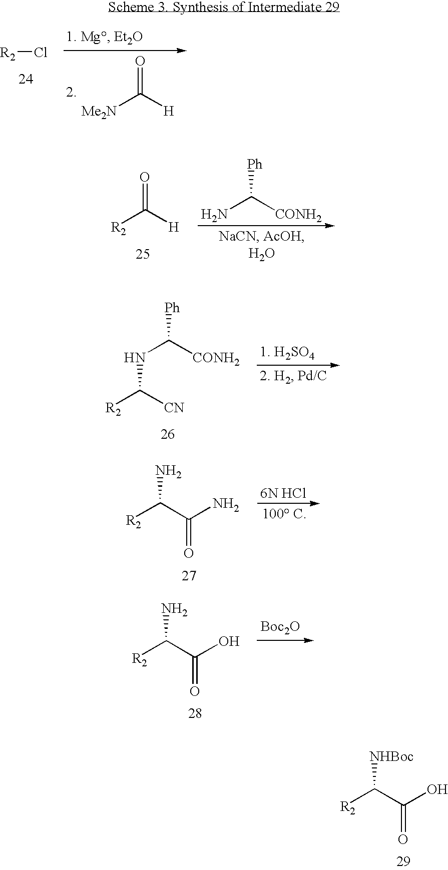

Scheme 3 above shows a synthesis of the deuterated N-Boc-tert-leucine intermediate 29, which is useful in Scheme 1. A deuterated Grignard reagent is generated in situ by reacting a deuterated 2-chloro-2-methylpropane 24 (such as commercially available 2-chloro-2-methylpropane-d₉) with magnesium metal. The Grignard reagent is treated with N,N-dimethylformamide according to the procedures described by Nazarski R B et al, Bull Soc Chim Belg 1992, 101:817-819 to afford the corresponding pivaldehyde 25. Treatment of the pivaldehyde 25 with (R)-phenylglycine amide, followed by reaction of the corresponding chiral imine with sodium cyanide according to the procedure described by Boesten, W H et al, Org Lett 2001, 3:1121-1124, affords the alpha-amino nitrile 26. Hydrolysis of nitrile 26 to the diamide followed by hydrogenolysis of the phenylacetamide group gives the alpha-amino amide 27. Finally, hydrolysis of the tert-leucine amide with 6N HCl gives the corresponding acid 28, which is then Boc-protected to form the desired N-Boc-tert-leucine reagent 29.

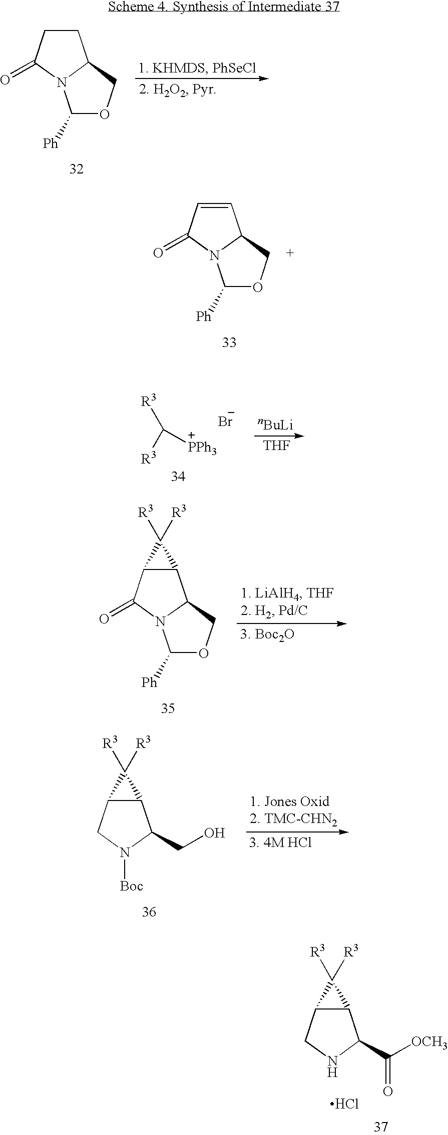

Scheme 4 shows the synthesis of a deuterated 3,4-dimethylcyclopropylproline 37, which is useful in Scheme 1. Treatment of the potassium enolate of (3R,7aS)-tetrahydro-3-phenyl-3H,5H-pyrrolol,2-coxaole-5-one (generated in situ by reacting commercially available (3R,7aS)-tetrahydro-3-phenyl-3H,5H-pyrrolo1,2-coxaole-5-one 32 with potassium hexamethyldisilazane) with phenyl selenium chloride followed by oxidation and elimination of the selenoxide according to the procedures described by Madalengoitia, J S et al, J Org Chem 1999, 64:547-555 affords the alpha, beta-unsaturated lactam 33. Treatment of the lactam 33 with a deuterated isopropylphosphonium ylide 34 (prepared in situ by from a deuterated isopropyl bromide (such as commercially available d₆-isopropyl bromide), see Braverman, S et al, J Am Chem Soc 1990, 112:5830-5837) using the procedure described by Ahmad, S et al, J Med Chem 2001, 44:3302-3310 affords the corresponding dimethylcyclopropyl lactam 35. Lactam 35 is converted to the requisite cyclopropylproline methyl ester 37 following the method described by Venkatraman, S et al, J Med Chem 2006, 49:6074-6086 affords.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al, Protective Groups in Organic Synthesis, 3^{rd} Ed., John Wiley and Sons (1999); Fieser L et al, Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I (e.g., including any of the formulae herein), or a pharmaceutically acceptable salt of said compound; and a pharmaceutically acceptable carrier acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as boceprevir. Such agents include those indicated as being useful in combination with boceprevir, including but not limited to, those described in WO 2006130666, WO 2006130628, WO 2007092616, and WO 2007092645.

Preferably, the second therapeutic agent is an agent useful in the treatment or prevention of a disease or condition selected from disorders associated with hepatitis C virus (HCV).

In one embodiment, the second therapeutic agent is selected from PEG-interferon alpha-2a, PEG-interferon alpha-2b, ribavirin, telapravir, nitazoxanide and combinations of two or more of the foregoing.

In a more specific embodiment, the second therapeutic agent is a combination of PEG-interferon alpha-2a and ribavirin.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al, (1966) Cancer Chemother Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 1 mg to about 8000 mg per treatment. In a more specific embodiment the range is from about 10 to 4000 mg, or from 20 to 1600 mg, or most specifically, from about 100 to 800 mg per treatment. Treatment typically is administered from one to three times daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the dosages of boceprevir being utilized in clinical trials.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of blocking the activity of HCV NS3/NS4A protease in an infected cell, comprising contacting such a cell with one or more compounds of Formula I herein.

According to another embodiment, the invention provides a method of treating a disease that is beneficially treated by boceprevir in a patient in need thereof comprising the step of administering to said patient an effective amount of a compound or a composition of this invention. Such diseases are well known in the art and are disclosed in, but not limited to the following patents and published applications: WO 2002008244, and WO 2003062265. Such diseases include, but are not limited to, disorders associated with hepatitis C virus (HCV).

In one particular embodiment, the method of this invention is used to treat a hepatitis C viral (HCV) infection in a patient in need thereof.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with boceprevir. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I and a second therapeutic agent for treatment of the following conditions (with the particular second therapeutic agent indicated in parentheses following the indication: hepatitis C (PEG-interferon, and ribavirin). (See clinical trials for SCH-503034 at http://clinicaltrials.gov/).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Diagnostic Methods and Kits

The present invention also provides kits for use to treat hepatitis C viral infection. These kits comprise (a) a pharmaceutical composition comprising a compound of Formula I or a salt thereof, wherein said pharmaceutical composition is in a container; and (b) instructions describing a method of using the pharmaceutical composition to treat hepatitis C viral infection.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

EVALUATION OF METABOLIC STABILITY. Certain in vitro liver metabolism studies have been described previously in the following references, each of which is incorporated herein in their entirety: Obach, R S, Drug Metab Disp, 1999, 27:1350; Houston, J B et al, Drug Metab Rev, 1997, 29:891; Houston, J B, Biochem Pharmacol, 1994, 47:1469; Iwatsubo, T et al, Pharmacol Ther, 1997, 73:147; and Lave, T, et al, Pharm Res, 1997, 14:152.

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich. The incubation mixtures are prepared according to Table 2:

TABLE 2
Reaction Mixture Composition for
Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

Determination of Metabolic Stability: Two aliquots of this reaction mixture are used for a compound of this invention. The aliquots are incubated in a shaking water bath at 37° C. for 3 minutes. The test compound is then added into each aliquot at a final concentration of 0.5 μM. The reaction is initiated by the addition of cofactor (NADPH) into one aliquot (the other aliquot lacking NADPH serves as the negative control). Both aliquots are then incubated in a shaking water bath at 37° C. Fifty microliters (50 μL) of the incubation mixtures are withdrawn in triplicate from each aliquot at 0, 5, 10, 20, and 30 minutes and combined with 50 μL of ice-cold acetonitrile to terminate the reaction. The same procedure is followed for boceprevir and 7-ethoxycoumarin, the positive control. Testing is done in triplicate.

Data analysis: The in vitro half-lives (t_{1/2}s) for test compounds are calculated from the slopes of the linear regression of % parent remaining (ln) vs incubation time relationship using the following formula:

in vitro t_{1/2}=0.693/k, where k=−[slope of linear regression of % parent remaining(ln) vs incubation time]

Data analysis is performed using Microsoft Excel Software.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a test compound in 100 mM potassium phosphate buffer (pH 7.4) was incubated at 37° C. in triplicate. Positive controls contain 1 μM of boceprevir instead of a test compound. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

### EXAMPLES

### Example 1

Synthesis of (S)-2-(Tert-butoxycarbonylamino)-3,3-di(methyl-d₃)-4-d₃-butanoic acid (56). Intermediate 56 was prepared as outlined in Scheme 6 below and as described below.

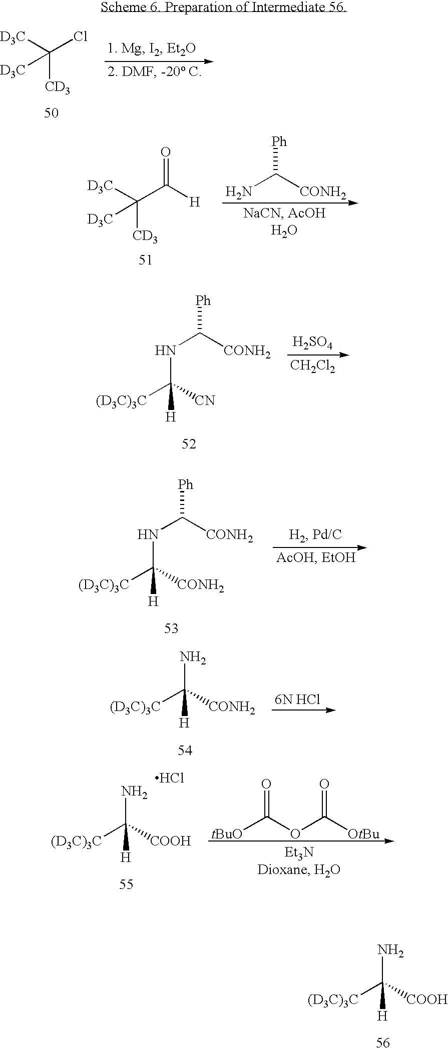

Synthesis of Pivalaldehyde-d₉ (51). In a 3-L 4-necked round bottom flask fitted with mechanical stirrer, reflux condenser, dropping funnel and thermometer were placed a few small crystals of iodine and then magnesium turnings (24.7 g, 1.03 mol). The bottom of the flask was heated with a heat gun until the iodine commenced to vaporize. The flask was allowed to cool while a solution of t-butyl chloride-dg 50 (100.0 g, 1.03 mol, Cambridge Isotopes, 98% isotopic purity) in anhydrous ether was placed in the dropping funnel. A portion of the solution of 50 in ether (3-5 mL) was added directly to the dry magnesium. More anhydrous ether (1 L) and a few small crystals of iodine were added, and the resulting mixture was heated for 0.5 hours (h) to initiate the reaction. The remainder of the solution of 50 in ether was added with stirring at a rate not faster than one drop per second. The mixture was allowed to reflux during the halide-ether addition and no external cooling was applied. The resulting mixture was heated at reflux for several hours until almost all magnesium had disappeared. The mixture was cooled to −20° C., and a solution of anhydrous DMF (73.0 g, 1.0 mol) in ether (100 mL) was added over a 35 minute (min) period at such a rate that the temperature of the reaction did not exceed −15° C. A second solution of anhydrous DMF (73.0 g, 1.0 mol) was then added quickly at −8° C. After an additional 5 min, hydroquinone (0.5 g) was added, stirring was stopped, the cooling bath was removed, and the mixture was left standing overnight at room temperature (rt) under nitrogen. The mixture was cooled to 5° C. and aqueous 4M HCl (600 mL) was added in portions to quench the reaction. The resulting mixture was diluted with water (400 mL), and the layers were separated. The aqueous layer was extracted with ether (3×200 mL), and the combined organic layers were dried (Na₂SO₄) and filtered. The filtrate was subjected to fractional distillation under an atmosphere of nitrogen to remove most of the ether. The residue was transferred to a small flask and fractional distillation was continued to afford 39.5 g (40% yield) of the desired compound 51 as a colorless oil at 65-75° C. Compound 51 was stored under nitrogen in the freezer.

Synthesis of (R)-2-((S)-1-Cyano-2,2-di(methyl-d₃)-3-d₃-propylamino)-2-phenylacetamide (52). To a stirred suspension of (R)-phenylglycine amide (60.7 g, 400 mmol) in water (400 mL) was added compound 51 (39.5 g, 415 mmol) at rt. This was followed by simultaneous addition of 30% aqueous NaCN solution (68.8 g, 420 mmol) and glacial acetic acid (25.4 g, 423 mmol) over 30 min, during which time the temperature of the reaction increased to 34° C. The mixture was stirred for 2 h at 30° C., followed by stirring at 70° C. for 20 h. After cooling to 30° C., the product was isolated by filtration. The solid was washed with water (500 mL) and dried under vacuum at 50° C. to afford the desired compound 52 (90.0 g, 88% yield) as a tan solid with [α]_{D}=−298° (c=1.0, CHCl₃).

Synthesis of (S)-2-((R)-2-Amino-2-oxo-1-phenylethylamino)-3,3-di(methyl-d₃)-4-d₃-butanamide (53). A solution of compound 52 (64.2 g, 252.4 mmol) in dichloromethane (500 mL) was added to conc. sulfuric acid (96%, 350 mL) at 15-20° C. through an addition funnel under the cooling of an ice bath. The resulting mixture was stirred at rt for 1 h then was poured onto ice and carefully neutralized by the addition of NH₄OH solution to pH of 9. The mixture was extracted with dichloromethane and the combined organic layers were washed with water, dried (Na₂SO₄), filtered, and concentrated in vacuo to afford the desired compound 53 (55.0 g, 80% yield) as a yellow foam with [α]_{D}=−140° (c=1.0, CHCl₃).

Synthesis of (S)-2-Amino-3,3-di(methyl-d₃)-4-d₃-butanamide (54). A mixture of compound 53 (77.0 g, 282.7 mmol), 10% Pd/C (approximately 50% water, 20 g) and acetic acid (50 mL) in ethanol (1.2 L) was subjected to hydrogenation at 30 psi, rt for several days until LCMS showed complete reaction. The resulting mixture was filtered through Celite and the solid washed with EtOAc. The filtrate was concentrated in vacuo, and the residue was diluted with water (1 L) then basified with 1M NaOH solution to pH of 9. The mixture was extracted with dichloromethane and the aqueous layer was concentrated in vacuo to half volume, saturated with solid NaCl, and extracted with THF. The combined extracts were dried (Na₂SO₄), filtered, and concentrated in vacuo. The residue was rinsed with toluene to remove remaining water, followed by trituration with dichloromethane to afford the desired compound 54 (38.0 g, 96% yield) as a white solid.

Synthesis of (S)-2-Amino-3,3-di(methyl-d₃)-4-d₃-butanamide hydrochloride (55). A mixture of compound 54 (31.0 g, 222.6 mmol) in 6M aqueous HCl solution (1.5 L) was heated at reflux for 24 h. The resulting mixture was concentrated in vacuo, leaving a solid, which was redissolved in water (500 mL) and washed with EtOAc (2×200 mL) to remove impurities from previous steps. The aqueous layer was then concentrated in vacuo, rinsed with toluene, and dried under vacuum at 50° C. to afford the HCl salt 55 (33.6 g, 85% yield) as a white solid.

Synthesis of (S)-2-(Tert-butoxycarbonylamino)-3,3-di(methyl-d₃)-4-d₃-butanoic acid (56). To a solution of compound 55 (1.00 g, 5.66 mmol) in a mixture of dioxane (10 mL) and water (10 mL) was added triethylamine (3.16 mL, 22.6 mmol) followed by di-tert-butyl dicarbonate (1.48 g, 6.79 mmol). The resulting mixture was stirred at rt for 6 h and then was extracted with heptane (2×20 mL). The aqueous fraction was cooled with an icebath, the pH was adjusted to 2 with 1M HCl, and then the fraction was extracted with ethyl acetate (3×50 mL). The combined extracts were dried (Na₂SO₄), filtered, and concentrated in vacuo to afford 56 (1.10 g, 81% yield) as a yellow oil.

### Example 2

Synthesis of (1S,2S,5S)-Methyl 6,6-di(methyl-d₃)-3-azabicyclo[3.1.0]hexane-2-carboxylate hydrochloride (63). Intermediate 63 was prepared as outlined in Scheme 7 below. Details of the synthesis follow.

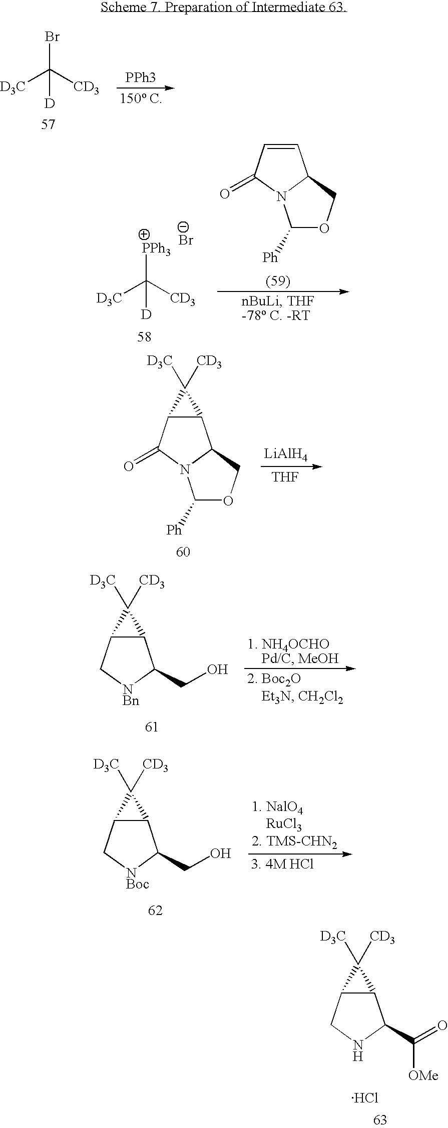

Synthesis of (Isopropyl-d₇)triphenylphosphonium (58). 2-Bromopropane-d₇ 57 (3.62 mL, 38.5 mmol, Aldrich, 98% isotopic purity) and triphenyl phosphine (10.09 g, 38.5 mmol) were stirred in a sealed pressure flask at 150° C. for 15 h. The reaction mixture was cooled to rt and the product crystallized from ethanol and diethyl ether. The product was filtered, washed with diethyl ether, and dried in vacuo to afford 58 as a white solid (9.95 g, 66% yield). MS (M-Br): 312.3.

Synthesis of Intermediate 60. To a solution of Wittig salt 58 (1.95 g, 4.98 mmol) in THF (15 mL) at −78° C. was added dropwise n-BuLi (2.5M in THF, 2.19 mL, 5.48 mmol). The reaction mixture was warmed to 0° C. and stirred for 30 min. The resulting red solution was cooled to −78° C. and a solution of lactam 59 (1.00 g, 4.98 mmol, commercially available from Enamine Building Blocks) in THF (10 mL) was added. The resulting mixture was stirred for 2 h at 0° C. followed by an additional 15 h at rt. The reaction was then quenched with saturated NaHCO₃ and extracted with ethyl acetate (3×50 mL). The organic layers were combined, dried (MgSO₄), filtered and concentrated in vacuo. The residue was purified via column chromatography (SiO₂, 10-30% EtOAc/heptane) to afford 60 (169 mg, 14% yield) as a pale yellow solid. MS (M+H): 250.2.

Synthesis of ((1S,2S,5S)-3-Benzyl-6,6-di(methyl-d₃)-3-azabicyclo[3.1.0]hexan-2-yl)methanol (61). To a solution of 60 (376 mg, 1.51 mmol) in THF (3 mL) at 0° C. was added LAH (2M in THF, 1.51 mL, 3.02 mmol). The reaction was heated to reflux for 3 h then cooled to 0° C. and quenched by dropwise addition of 10% aqueous KHSO₄. The resulting slurry was diluted with ethyl acetate and filtered (washing the filter cake with ethyl acetate (2×10 mL). The filtrate was diluted with water (20 mL) and extracted with ethyl acetate (3×30 mL). The combined organic layers were washed with brine, dried (MgSO₄), and concentrated in vacuo to afford 61 (350 mg, 98% yield) which was used without further purification. MS (M+H): 238.3.

Synthesis of (1S,2S,5S)-Tert-butyl 2-(hydroxymethyl)-6,6-di(methyl-d₃)-3-azabicyclo[3.1.0]hexane-3-carboxylate (62). To a solution of 61 (350 mg, 1.48 mmol) in methanol (15 mL) was added ammonium formate (571 mg, 9.06 mmol) followed by 10% palladium on carbon (70 mg, 20 wt. %). The resulting mixture was heated to reflux, taking precautions to limit ammonium formate sublimation inside the condenser. After stirring at reflux for 2 h, the mixture was cooled to rt and filtered through Celite. The Celite pad was washed with methanol (2×10 mL), followed by dichloromethane (2×20 mL). The resulting solution was then concentrated in vacuo to afford the desired deuterated amino alcohol. This material (approximately 1.48 mmol) was dissolved in dichloromethane (5 mL), and triethylamine (273 μL, 1.96 mmol), followed by di-tert-butyl dicarbonate (428 mg, 1.96 mmol), was added. The reaction mixture was stirred at rt for 15 h then was diluted with 1M HCl (15 mL) and extracted with dichloromethane (3×30 mL). The combined organic layers were washed with brine, dried (Na₂SO₄), filtered, and then concentrated in vacuo. The resulting residue was purified by column chromatography (SiO₂, 0-20% EtOAc/heptane) to afford 62 (311 mg, 83%—2 step yield). MS (M-^{t}Bu): 192.3.

Synthesis of (1S,2S,5S)-Methyl 6,6-di(methyl-d₃)-3-azabicyclo[3.1.0]hexane-2-carboxylate hydrochloride (63). To a stirred solution of 62 (311 mg, 1.26 mmol) in ethyl acetate (10 mL) and acetonitrile (10 mL) was added a solution of ruthenium trichloride monohydrate (5.50 mg, 0.0252 mmol) and sodium periodate (2.16 g, 10.0 mmol) in water (15 mL). The mixture was stirred at rt for 1 h, then was filtered through Celite. The Celite pad was washed with ethyl acetate (3×5 mL) and the resulting solution was concentrated in vacuo. The resulting residue was diluted with 1M HCl (10 mL) and extracted with ethyl acetate (3×20 mL). The organic layers were combined, washed with 1M HCl, dried (Na₂SO₄), filtered and concentrated in vacuo to afford the crude deuterated acid as a dark tan solid. This acid (313 mg, 1.20 mmol) was dissolved in a mixture of benzene (5.0 mL) and methanol (0.50 mL) and a 2M solution of trimethylsilyl diazomethane in hexanes (780 μL, 1.56 mmol) was added dropwise. The yellow solution was stirred at rt for 15 h and was subsequently quenched by the dropwise addition of acetic acid until effervescence ceased. The reaction was then concentrated in vacuo with several repeated heptane dilutions/concentrations to remove excess acetic acid. The resulting residue was purified by column chromatography (SiO₂, 0-30% EtOAc/heptane) to afford the pure deuterated methyl ester of 63 (154 mg). To this material was added a 4M solution of HCl in dioxane (5.0 mL) and the resulting solution was stirred at rt for 2 h. The reaction was then concentrated in vacuo to afford the pure hydrochloride salt 63 (128 mg, 41%-3 step yield) as a colorless solid. MS (M+H): 176.2 (free amine).

### Example 3

Synthesis of (1S,2S,5S)—N-(4-amino-1-cyclobutyl-3,4-dioxobutan-2-yl)-3-((S)-2-(3-(tert-butyl-d₉)ureido)-3,3-di(methyl-d₃)-4-d₃-butanoyl)-6,6-dimethyl-3-azabicyclo[3.1.0]hexane-2-carboxamide (109). Compound 109 was prepared as a mixture of 109 and its diastereomer as outlined in Scheme 8 below. Details of the synthesis are set forth below.

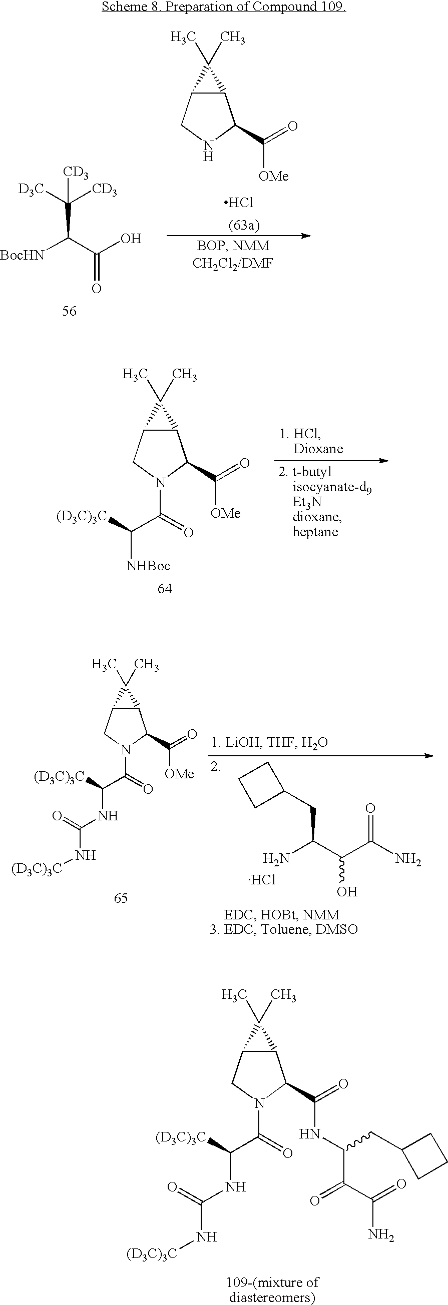

Synthesis of (1S,2S,5S)-Methyl 3-((S)-2-(tert-butoxycarbonylamino)-3,3-di(methyl-d₃)-4-d₃-butanoyl)-6,6-dimethyl-3-azabicyclo[3.1.0]hexane-2-carboxylate (64). To a solution of 56 (258 mg, 1.07 mmol) and amine hydrochloride salt 63a (265 mg, 1.29 mmol, see Venkatraman, S et al, J Med Chem, 2006, 49: 6074-6086 for the preparation) in CH₂Cl₂/DMF (6 mL, 1:1) at 0° C. was added N-methyl morpholine (353 μL, 3.21 mmol) and benzotriazole-1-yl-oxy-tris-(dimethylamino)-phosphonium hexafluorophosphate (“BOP reagent”, 571 mg, 1.29 mmol). The reaction mixture was stirred at rt for 15 h, diluted with 1M HCl, and extracted with CH₂Cl₂ (3×50 mL). The combined organic layers were washed with 1M HCl, saturated NaHCO₃ and brine, dried (MgSO₄), filtered, and then concentrated in vacuo. The resulting residue containing the desired deuterated methyl ester 64 was used without further purification. MS (M+H): 392.4.

Synthesis of (1S,2S,5S)-Methyl 3-((S)-2-(3-(tert-butyl-d₉)-ureido)-3,3-di(methyl-d₃)-4-d₃-butanoyl)-6,6-dimethyl-3-azabicyclo[3.1.0]hexane-2-carboxylate (65). A solution of 64 (approximately 1.29 mmol) in 4M HCl in dioxane (7 mL) was stirred at rt for 3 h, and then was concentrated in vacuo to afford the corresponding amine hydrochloride salt of 64. This material was dissolved in dioxane (1.00 mL) and triethylamine (450 μL, 3.22 mmol) was added. The mixture was cooled to −78° C. and t-butyl isocyanate-d₉ (324 mg, 3.00 mmol, in 20 mL heptane/dioxane 1:1, synthesized according to Giribone, D et al, J. Labelled Compd Radioparm., 2000, 43: 933-941, from t-butylamine-d₉, (CDN Isotopes 99% isotopic purity)) was added. The reaction mixture was stirred at rt for 15 h, concentrated in vacuo, diluted with 1M HCl, then extracted with CH₂Cl₂ (3×50 mL). The combined organic layers were washed with 1M HCl, saturated NaHCO₃ and brine, dried (MgSO₄), filtered, and concentrated in vacuo. The resulting residue was purified by column chromatography (SiO₂, 0-20% acetone/heptane) to afford 65 (125 mg, 30%-3 steps) as a dry white foam. MS (M+Na): 422.4.

Synthesis of (1S,2S,5S)—N-(4-Amino-1-cyclobutyl-3,4-dioxobutan-2-yl)-3-((S)-2-(3-(tert-butyl-d₉)ureido)-3,3-di(methyl-d₃)-4-d₃-butanoyl)-6,6-dimethyl-3-azabicyclo[3.1.0]hexane-2-carboxamide (109). To a solution of 65 (125 mg, 0.313 mmol) in a mixture of THF/H₂O (4 mL, 1:1) was added lithium hydroxide (11.0 mg, 0.469 mmol). The reaction mixture was stirred for 3 h, quenched with 1M HCl, and concentrated in vacuo to remove THF. The resulting aqueous solution was extracted with EtOAc (3×30 mL) and the combined organic layers were dried (Na₂SO₄), filtered and concentrated in vacuo. The residue was dissolved in a mixture of CH₂Cl₂/DMF (4 mL, 1:1) and the resulting solution was cooled to −20° C. To this solution was added amine hydrochloride salt 66 (86.0 mg, 0.411 mmol, see Venkatraman, S et al, J Med Chem, 2006, 49: 6074-6086 for preparation), 1-ethyl-3-(3-dimethylaminopropyl) carbodiimide hydrochloride, (“EDC”, 98.0 mg, 0.513 mmol), hydroxybenzotriazole (“HOBt”, 69.0 mg, 0.513 mmol), and N-methyl morpholine (150 μL, 1.37 mmol). The reaction was stirred at −20° C. for 48 h and concentrated in vacuo. The resulting residue was diluted with 1M HCl and this solution was extracted with EtOAc (3×30 mL). The combined organic layers were washed with 1M HCl, saturated NaHCO₃ and brine, dried (MgSO₄), filtered and concentrated in vacuo to provide a light-yellow foam. To a solution of this material in a mixture of toluene/DMSO (6 mL, 1:1) at 0° C. was added EDC (533 mg, 2.78 mmol) and dichloroacetic acid (115 μL, 1.39 mmol). The reaction was stirred at rt for 4 h, then diluted with saturated NaHCO₃ and extracted with CH₂Cl₂ (3×30 mL). The combined organic layers were washed with saturated NaHCO₃, 1M HCl and brine, dried (MgSO₄), filtered, and concentrated in vacuo. The resulting residue was purified by column chromatography (SiO₂, 0-30% acetone/heptane) to afford 109 (62.0 mg, 37% over 3 steps) as a white solid which was a mixture of diastereomers. ¹H NMR (DMSO-d₆, 400 MHz): δ 8.27 (d, 0.8H, J=7.3 Hz), 8.17 (d, 0.2H, J=7.8 Hz), 8.02 (s, 0.8H), 7.97 (s, 0.2H), 7.76 (broad s, 1.0H), 7.34 (s, 0.5H), 6.90 (s, 0.4H), 5.94 (s, 0.8H), 5.93 (s, 0.2H), 5.87-5.79 (m, 1H), 5.00-4.90 (m, 0.8H), 4.90-4.81 (m, 0.2H), 4.28 (s, 0.8H), 4.27 (s, 0.2H), 4.16-4.06 (m, 1H), 4.00-3.90 (m, 1H), 3.80-3.69 (m, 1H), 2.57-2.42 (m, 0.8H), 2.40-2.29 (m, 0.2H), 2.04-1.89 (m, 2H), 1.82-1.68 (m, 3H), 1.68-1.52 (m, 3H), 1.45-1.39 (m, 1H), 1.31-1.18 (m, 1H), 1.03-0.97 (m, 3H), 0.89-0.80 (m, 3H). MS (M+Na): 560.3; (M+H) 538.5.

### Example 4

Synthesis of (1S,2S,5S)—N-(4-amino-1-cyclobutyl-3,4-dioxobutan-2-yl)-3-((S)-2-(3-(tert-butyl-d₉)ureido)-3,3-di(methyl-d₃)-4-d₃-butanoyl)-6,6-di(methyl-d₃-azabicyclo[3.1.0]hexane-2-carboxamide (103). Compound 103 was prepared as a mixture of 103 and its diastereomer as generally outlined in Scheme 8 above. Details of the synthesis are set forth below.

Synthesis of (1S,2S,5S)-Methyl 3-((S)-2-(tert-butoxycarbonylamino)-3,3-di(methyl-d₃)-4-d₃-butanoyl)-6,6-di(methyl-d₃)-3-azabicyclo[3.1.0]hexane-2-carboxylate (38, R²═(CD₃)₃C; R³═CD₃). To a solution of 56 (121 mg, 0.506 mmol, see Example 1) and amine hydrochloride salt 63 (128 mg, 0.607 mmol, see Example 2) in CH₂Cl₂/DMF (3 mL, 1:1) at 0° C. was added N-methyl morpholine (167 μL, 1.52 mmol) and BOP reagent (269 mg, 0.607 mmol). The reaction was stirred at rt for 15 h, diluted with 1M HCl, and extracted with CH₂Cl₂ (3×30 mL). The combined organic layers were washed with 1M HCl, saturated NaHCO₃ and brine, dried (MgSO₄), filtered, and concentrated in vacuo. The resulting residue was purified by column chromatography (SiO₂, 0-20% acetone/heptane) to afford 38 (R²═(CD₃)₃C; R³═CD₃) (112 mg, 56% yield). MS (M+Na): 420.4; (M+H): 398.3.

Synthesis of (1S,2S,5S)-Methyl 3-((S)-2-(3-(tert-butyl-d₉)-ureido)-3,3-di(methyl-d₃)-4-d₃-butanoyl)-6,6-di(methyl-d₃)-3-azabicyclo[3.1.0]hexane-2-carboxylate (39, R¹═(CD₃)₃C; R²═(CD₃)₃C; R³═CD₃). A solution of 38 (R²═(CD₃)₃C; R³═CD₃) (112 mg, 0.282 mmol) in 4M HCl in dioxane (5 mL) was stirred at rt for 3 h then concentrated in vacuo. The resultant amine hydrochloride salt was dissolved in dioxane (300 μL) and triethylamine (197 mL, 1.40 mmol) was added with stirring. The mixture was cooled to −78° C., tert-butyl isocyanate-dg (130 mg, 1.20 mmol, in 10 mL heptane/dioxane 1:1, see above) was added and the reaction mixture was stirred at rt for 15 h. The resulting mixture was concentrated in vacuo, diluted with 1 M HCl, and extracted with CH₂Cl₂ (3×30 mL). The combined organic layers were washed with 1M HCl, saturated NaHCO₃ and brine, dried (MgSO₄), filtered, and concentrated in vacuo. The resulting residue was purified by column chromatography (SiO₂, 0-20% acetone/heptane) to afford 39 (R¹═(CD₃)₃C; R²═(CD₃)₃C; R³═CD₃) (74 mg, 65%) as a dry white foam. MS (M+Na): 428.5.

Synthesis of (1S,2S,5S)—N-(4-amino-1-cyclobutyl-3,4-dioxobutan-2-yl)-3-((S)-2-(3-(tert-butyl-d₉)ureido)-3,3-di(methyl-d₃)-4-d₃-butanoyl)-6,6-di(methyl-d₃)-3-azabicyclo[3.1.0]hexane-2-carboxamide (103). To a solution of 39 (R¹═(CD₃)₃C; R²═(CD₃)₃C; R³═CD₃) (74 mg, 0.182 mmol) in a mixture of THF/H₂O (2 mL, 1:1) was added lithium hydroxide (7.00 mg, 0.274 mmol). The reaction was stirred for 3 h, quenched with 1M HCl and concentrated under reduced pressure to remove THF. The resulting aqueous solution was extracted with EtOAc (3×10 mL) and the combined organic layers were dried (Na₂SO₄), filtered and concentrated in vacuo. The resulting residue was dissolved in a mixture of CH₂Cl₂/DMF (2 mL, 1:1) and cooled to −20° C.

To this solution was added the amine hydrochloride salt 66 (46.0 mg, 0.218 mmol, see Example 3 above), EDC (52.0 mg, 0.273 mmol), HOBt (37.0 mg, 0.273 mmol), and N-methyl morpholine (80.0 μL, 0.728 mmol). The reaction was stirred at −20° C. for 48 h then was concentrated in vacuo. The resulting residue was diluted with 1 M HCl and extracted with EtOAc (3×10 mL). The combined organic layers were washed with 1M HCl, saturated NaHCO₃ and brine, dried (MgSO₄), filtered, and concentrated in vacuo to provide a light yellow foam. This material was dissolved in a mixture of toluene/DMSO (3 mL, 1:1) at 0° C. followed by addition of EDC (305 mg, 1.59 mmol) and dichloroacetic acid (65.0 μL, 0.795 mmol). The reaction was stirred at rt for 4 h, then diluted with saturated NaHCO₃ and extracted with CH₂Cl₂ (3×10 mL). The combined organic layers were washed with saturated NaHCO₃, 1M HCl and brine, dried (MgSO₄), filtered, and concentrated in vacuo. The resulting residue was purified by column chromatography (SiO₂, 0-30% acetone/heptane) to afford 103 (20.2 mg, 23%-3 steps) as a white solid and as a mixture of diastereomers. ¹H NMR (DMSO-d₆, 400 MHz) δ 8.27 (d, 0.7H, J=7.3 Hz), 8.17 (d, 0.3H, J=7.8 Hz), 8.02 (s, 0.7H), 7.97 (s, 0.3H), 7.76 (br. s, 1.0H), 7.34 (s, 0.6H), 6.90 (s, 0.6H), 5.94 (s, 0.7H), 5.93 (s, 0.3H), 5.87-5.79 (m, 1H), 5.00-4.90 (m, 0.7H), 4.90-4.81 (m, 0.3H), 4.28 (s, 0.7H), 4.27 (s, 0.3H), 4.16-4.06 (m, 1H), 4.00-3.90 (m, 1H), 3.80-3.69 (m, 1H), 2.57-2.42 (m, 0.7H), 2.40-2.29 (m, 0.3H), 2.04-1.89 (m, 2H), 1.82-1.68 (m, 3H), 1.68-1.52 (m, 3H), 1.45-1.39 (m, 1H), 1.31-1.18 (m, 1H). MS (M+Na): 566.5; (M+H) 544.5.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of the formula: 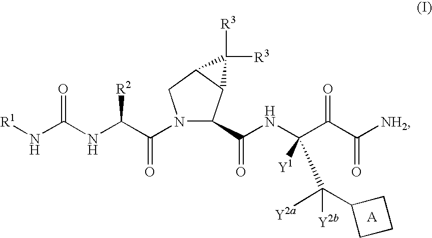
or a pharmaceutically acceptable salt thereof, wherein:
Ring A is a cyclobutyl ring having zero to seven deuterium atoms;
each of R¹ and R² is independently —C(CH₃)₃, wherein 1 to 9 hydrogen atoms are optionally replaced with deuterium atoms;
each R³ is independently selected from —CH₃, —CH₂D, —CHD₂, and —CD₃;
each Y is independently selected from hydrogen and deuterium; and
at least one Y is deuterium when R¹ and R² are simultaneously —C(CH₃)₃, R³ is —CH₃, and ring A has zero deuterium atoms.

2. The compound of claim 1, wherein R¹ is selected from —C(CH₃)₃ and —C(CD₃)₃.

3. The compound of claim 2, wherein R² is selected from —C(CH₃)₃ and —C(CD₃)₃.

4. The compound of claim 3, wherein each R³ is the same.

5. The compound of claim 4, wherein each R³ is independently selected from —CH₃ and —CD₃.

6. The compound of claim 5, wherein at least one of R¹ and R² is —C(CD₃)₃, or each R³ is —CD₃.

7. The compound of claim 6, wherein Ring A has zero or seven deuterium atoms.

8. The compound of claim 7, wherein each Y² is the same.

9. The compound of claim 8, wherein:
each of R¹ and R² is —C(CD₃)₃;
each R³ is the same and is selected from —CD₃ and —CH₃; and
Ring A is selected from a cyclobutyl ring having 0 deuterium atoms and a cyclobutyl ring having 7 deuterium atoms.

10. The compound of claim 1 selected from any one of the compounds set forth in the table below:

| Compound | R¹ | R² | each R³ | Y¹ | each Y² | Ring A |
|---|---|---|---|---|---|---|
| 100 | C(CD₃)₃ | C(CD₃)₃ | CD₃ | D | D | D₇ |
| 101 | C(CD₃)₃ | C(CD₃)₃ | CD₃ | D | D | H₇ |
| 102 | C(CD₃)₃ | C(CD₃)₃ | CD₃ | D | H | H₇ |
| 103 | C(CD₃)₃ | C(CD₃)₃ | CD₃ | H | H | H₇ |
| 104 | C(CD₃)₃ | C(CD₃)₃ | CD₃ | H | H | D₇ |
| 105 | C(CD₃)₃ | C(CD₃)₃ | CD₃ | D | H | D₇ |
| 106 | C(CD₃)₃ | C(CD₃)₃ | CH₃ | D | D | D₇ |
| 107 | C(CD₃)₃ | C(CD₃)₃ | CH₃ | H | D | D₇ |
| 108 | C(CD₃)₃ | C(CD₃)₃ | CH₃ | H | H | D₇ |
| 109 | C(CD₃)₃ | C(CD₃)₃ | CH₃ | H | H | H₇ |
| 110 | C(CD₃)₃ | C(CD₃)₃ | CH₃ | H | D | H₇ |
| 111 | C(CD₃)₃ | C(CD₃)₃ | CH₃ | D | D | H₇ |
| 112 | C(CD₃)₃ | C(CD₃)₃ | CH₃ | D | H | H₇ |
| 113 | C(CD₃)₃ | C(CH₃)₃ | CD₃ | D | D | D₇ |
| 114 | C(CD₃)₃ | C(CH₃)₃ | CD₃ | H | D | D₇ |
| 115 | C(CD₃)₃ | C(CH₃)₃ | CD₃ | H | H | D₇ |
| 116 | C(CD₃)₃ | C(CH₃)₃ | CD₃ | D | D | H₇ |
| 117 | C(CD₃)₃ | C(CH₃)₃ | CD₃ | H | D | H₇ |
| 118 | C(CD₃)₃ | C(CH₃)₃ | CD₃ | D | H | H₇ |
| 119 | C(CD₃)₃ | C(CH₃)₃ | CD₃ | H | H | H₇ |
| 120 | C(CH₃)₃ | C(CD₃)₃ | CD₃ | D | D | D₇ |
| 121 | C(CH₃)₃ | C(CD₃)₃ | CD₃ | H | D | D₇ |
| 122 | C(CH₃)₃ | C(CD₃)₃ | CD₃ | H | H | D₇ |
| 123 | C(CH₃)₃ | C(CD₃)₃ | CD₃ | D | H | D₇ |
| 124 | C(CH₃)₃ | C(CD₃)₃ | CD₃ | D | D | H₇ |
| 125 | C(CH₃)₃ | C(CD₃)₃ | CD₃ | H | D | H₇ |
| 126 | C(CH₃)₃ | C(CD₃)₃ | CD₃ | H | H | H₇ |
| 127 | C(CH₃)₃ | C(CD₃)₃ | CD₃ | D | H | H₇ |
| 128 | C(CH₃)₃ | C(CH₃)₃ | CD₃ | D | D | D₇ |
| 129 | C(CH₃)₃ | C(CH₃)₃ | CD₃ | D | H | D₇ |
| 130 | C(CH₃)₃ | C(CH₃)₃ | CD₃ | H | D | D₇ |
| 131 | C(CH₃)₃ | C(CH₃)₃ | CD₃ | D | D | H₇ |
| 132 | C(CH₃)₃ | C(CH₃)₃ | CD₃ | H | D | H₇ |
| 133 | C(CH₃)₃ | C(CH₃)₃ | CD₃ | H | H | D₇ |
| 134 | C(CH₃)₃ | C(CH₃)₃ | CD₃ | H | H | H₇ |
| 135 | C(CH₃)₃ | C(CH₃)₃ | CD₃ | D | H | H₇ |
| 136 | C(CD₃)₃ | C(CH₃)₃ | CH₃ | D | D | D₇ |
| 137 | C(CD₃)₃ | C(CH₃)₃ | CH₃ | D | H | D₇ |
| 138 | C(CD₃)₃ | C(CH₃)₃ | CH₃ | H | D | D₇ |
| 139 | C(CD₃)₃ | C(CH₃)₃ | CH₃ | D | D | H₇ |
| 140 | C(CD₃)₃ | C(CH₃)₃ | CH₃ | H | D | H₇ |
| 141 | C(CD₃)₃ | C(CH₃)₃ | CH₃ | H | H | D₇ |
| 142 | C(CD₃)₃ | C(CH₃)₃ | CH₃ | D | H | H₇ |
| 143 | C(CD₃)₃ | C(CH₃)₃ | CH₃ | H | H | H₇ |
| 144 | C(CH₃)₃ | C(CD₃)₃ | CH₃ | D | D | D₇ |
| 145 | C(CH₃)₃ | C(CD₃)₃ | CH₃ | D | H | D₇ |
| 146 | C(CH₃)₃ | C(CD₃)₃ | CH₃ | H | D | D₇ |
| 147 | C(CH₃)₃ | C(CD₃)₃ | CH₃ | D | D | H₇ |
| 148 | C(CH₃)₃ | C(CD₃)₃ | CH₃ | H | D | H₇ |
| 149 | C(CH₃)₃ | C(CD₃)₃ | CH₃ | H | H | D₇ |
| 150 | C(CH₃)₃ | C(CD₃)₃ | CH₃ | D | H | H₇ |
| 151 | C(CH₃)₃ | C(CD₃)₃ | CH₃ | H | H | H₇ |
| 152 | C(CH₃)₃ | C(CH₃)₃ | CH₃ | D | D | D₇ |
| 153 | C(CH₃)₃ | C(CH₃)₃ | CH₃ | D | H | D₇ |
| 154 | C(CH₃)₃ | C(CH₃)₃ | CH₃ | H | D | D₇ |
| 155 | C(CH₃)₃ | C(CH₃)₃ | CH₃ | D | D | H₇ |
| 156 | C(CH₃)₃ | C(CH₃)₃ | CH₃ | H | D | H₇ |
| 157 | C(CH₃)₃ | C(CH₃)₃ | CH₃ | H | H | D₇ |

wherein D₇ represents a cyclopentyl ring having 7 deuterium atoms; and H₇ represents a cyclopentyl ring having 0 deuterium atoms or a pharmaceutically acceptable salt of any of the foregoing.

11. The compound of claim 10 selected from: 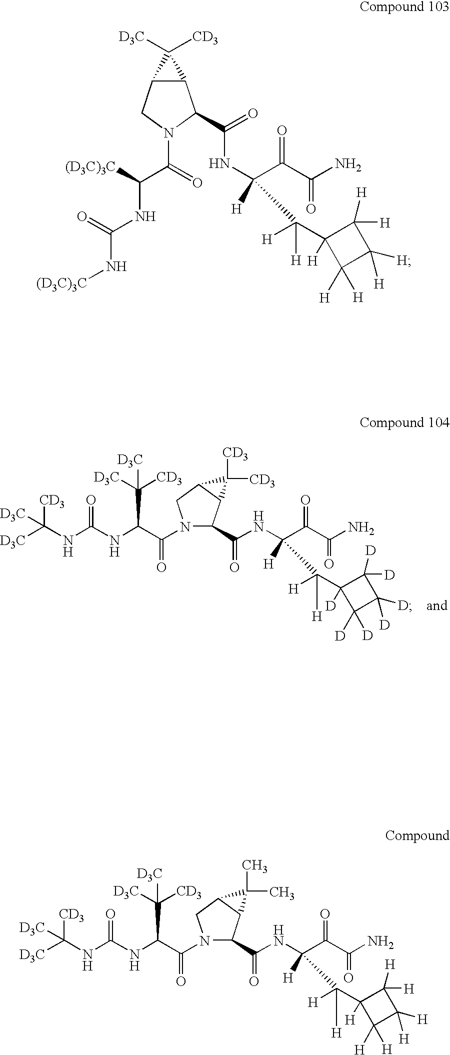
109 or a pharmaceutically acceptable salt of any of the foregoing.

12. The compound of claim 1, wherein any atom not designated as deuterium is present at its natural isotopic abundance.

13. A pyrogen-free pharmaceutical composition comprising a compound of claim 1 and a pharmaceutically acceptable carrier.

14. The composition of claim 13, further comprising a second therapeutic agent useful in the treatment or prevention of a hepatitis C virus (HCV) infection.

15. The composition of claim 14, wherein the second therapeutic agent is selected from PEG-interferon alpha-2a, PEG-interferon alpha-2b, ribavirin, telapravir, nitazoxanide and combinations of any two or more of the foregoing.

16. The composition of claim 15, wherein the second therapeutic agent is a combination of PEG-interferon alpha-2a and ribavirin.

17. A method of treating hepatitis C viral (HCV) infection in a patient comprising the step of administering to the patient an effective amount of a compound of claim 1 or a composition of claim 13.

18. The method of claim 17, further comprising the step of co-administering to the patient in need thereof a second therapeutic agent selected from PEG-interferon alpha-2a, PEG-interferon alpha-2b, ribavirin, telapravir, nitazoxanide and combinations of any two or more of the foregoing.

19. The method of claim 18, wherein the second therapeutic agent is a combination of PEG-interferon alpha-2a and ribavirin.
